(19) United States
(12) Patent Application Publication
(10) Pub. No.: US 2025/0213623 A1
(43) Pub. Date: Jul. 3, 2025

(54) METHOD OF PREPARATION OF A BIOLOGICAL THERAPEUTIC PRODUCT BASED ON PERINATAL TISSUE, AND THE PRODUCT

(71) Applicant: PRIMECELL BIOSCIENCE, A.S., Ostrava (CZ)

(72) Inventors: Serhiy FOROSTYAK, Praha 8 (CZ); Jana DEDKOVA, Zelechovice nad Drevnici (CZ); Michal ZAHRADNICEK, Praha 1 (CZ); Lucia PISTOROVA, Vresina (CZ); Petr KOSKA, Pribice 142 (CZ)

(21) Appl. No.: 18/847,461

(22) PCT Filed: Mar. 14, 2023
(86) PCT No.: PCT/CZ2023/050013

§ 371 Date: Sep. 16, 2024

(30) Foreign Application Priority Data
Mar. 15, 2022 (CZ) PV 2022-116

(51) Int. Cl. A61K 35/50; A61K 9/19

(52) U.S. Cl. CPC A61K 35/50; A61K 9/19

(57) ABSTRACT

The present invention provides a method of preparation of a biological therapeutic product based on perinatal tissue, characterized in that the method includes the steps of:
a) cleaning of the starting perinatal tissue and optionally its resection and/or shaping into sections whose dimensions do not exceed 50 mm in any direction; and allowing the tissue to dry for at least 15 minutes after the cleaning;
b) cryo-grinding of the perinatal tissue in resealable surgical steel capsules with surgical steel grinding balls;
c) homogenization of the ground perinatal tissue using an ultrasonic homogenizer;
d) transferring the ground and homogenized perinatal tissue into a primary packaging, with constant stirring;
e) lyophilization of the ground and homogenized perinatal tissue in the primary packaging;
f) sterilization of the lyophilized ground and homogenized perinatal tissue in the primary packaging by gamma radiation at a dose of 5 to 20 kGy;
wherein the entire process is carried out without the presence of antibiotics and antimycotics.

### FIELD OF ART

The present invention relates to a method of producing a biological therapeutic product, suitable in particular for the treatment of musculoskeletal and connective tissue and skin diseases. The starting material for the production of the product is human amniotic membrane, chorion, umbilical cord and other perinatal tissues.

### BACKGROUND ART

Osteoarthritis (OA) is a degenerative arthropathy characterized by episodes of acute pain. OA is one of the most common chronic musculoskeletal diseases. Current pharmacological treatment of OA depends on whether it is an acute or chronic form of the disease. Therapy of the acute OA, when the pain temporarily becomes high-intensity pain, consists in the administration of analgesics, non-steroidal anti-inflammatory drugs (NSAIDs) or intra-articular corticosteroid injections. In the chronic osteoarthritis, alleviation of symptoms is mainly based on the administration of so-called symptomatic slow-acting drugs for OA (SYSADOA), which include precursors of cartilage extracellular matrix, such as hyaluronic acid (HA), glucosamine or chondroitin sulfate; or cytokine modulators such as diacerein, piascledin or matrix metalloproteinase (MMP) inhibitors.

Treatment of OA using intra-articular injections of amniotic membrane and/or umbilical cord particles has been proposed recently. Currently, several procedures for the production of placental based product for injection already exist.

In WO2014143990A1, both amnion and chorion are purified using antibiotics and then processed with trypsin (enzyme causing epithelial cell necrosis) or collagenase at 37° C. The material is repeatedly frozen and thawed during the process. The final product is not sterilized by final γ-sterilization. At the end of the process, the mixture is combined with hyaluronic acid, collagen, fibrin or gelatin containing multipotent cells, and supplied in a frozen state.

US20170258727A1 describes methods of preparing powders from fetal tissue and methods of using this powder product. The product uses a mixture of amnion, chorion and umbilical cord tissue. During sampling, the tissue is exposed to antibiotics (amphotericin, ciprofloxacin). In this production method, the tissue is first frozen, then lyophilized, and then ground, which leads to uneven grinding and mechanical degradation of the tissue. In the lyophilization step, pre-drying takes place first at a temperature of −5 to 25° C., then primary lyophilization takes place at temperatures of 0 to −100° C. and finally secondary lyophilization takes place at 25° C. In some cases, HEPES buffer, glycerol or propylene glycol are used during preparation. In some cases, the product was directly exposed to liquid nitrogen.

US20190374584A1 describes methods of production and use of micronized composites made from amnion and chorion and their pharmaceutical use. The placenta is transported on dry ice after collection. The amnion is cleaned using sterile gauze and solutions containing Triton X-100 and antibiotics (streptomycin, gentamicin, polymixn B, bacitracin). For dehydration, chemical dehydration (using dimethyl sulfoxide, acetone, ethanol, isopropanol) is used in combination with freeze drying. The dehydration step is followed by micronization on an oscillating mill. No final sterilization is carried out during the production of this product.

Current methods of processing the amniotic membrane into powder materials suitable for protecting and supporting the healing of damaged tissues, in which the properties of the membrane are preserved, require the use of antibiotics during the processing, although the procedure is always carried out aseptically under GMP conditions. All existing methods use chemicals or large temperature fluctuations to prepare the final product; this can have a negative effect on the integrity of the therapeutically active proteins. The present invention aims to provide an amniotic membrane processing procedure that allows the production of micronized material for injection without the need to add any chemicals or antibiotics, while minimizing temperature fluctuations in order to maximize the protection of proteins that can affect the biological and mechanical properties of the amniotic membrane-based material.

### DISCLOSURE OF THE INVENTION

The present invention provides a method for preparing a biological therapeutic product based on perinatal tissue, said method including the steps:
a) cleaning of the starting perinatal tissue and optionally its resection and/or shaping into sections whose dimensions do not exceed 50 mm in any direction; and allowing the tissue to dry for at least 15 minutes after the cleaning;
b) cryo-grinding of the perinatal tissue in resealable surgical steel capsules with surgical steel grinding balls, preferably made of surgical steel No. 1.4034 (stainless steel equivalent to the American Iron and Steel Institute 420C standard);
c) homogenization of the ground perinatal tissue using an ultrasonic homogenizer, preferably using a vortex;
d) transferring the ground and homogenized perinatal tissue into a primary packaging, with constant stirring;
e) lyophilization of the ground and homogenized perinatal tissue in the primary packaging;
f) sterilization of the lyophilized material, i.e. of the ground and homogenized perinatal tissue forming the biological therapeutic product, in the primary packaging by gamma radiation at a dose of 5 to 20 kGy;
wherein the entire process is carried out without the presence of antibiotics and antimycotics.

The starting perinatal tissue is in accordance with the legislation (for example, with the Czech Act No. 296/2008 Coll. on tissues and cells and Decree 422/2008 Coll. on establishing further requirements for ensuring the quality and safety of human tissues and cells intended for human use) obtained from informed donors after cesarean delivery. The starting perinatal tissue is mainly the amniotic membrane or the umbilical cord, which are separated from the donor placenta. The amnion is the internal fetal envelope surrounding the amniotic cavity which is filled with amniotic fluid. The amniotic epithelium passes through the umbilical cord (lat. chorda umbilicalis) to the periderm. The donor's blood is tested for hepatitis B (HBsAg, anti HBc), hepatitis C (anti HCV), HIV 1 and HIV 2 (anti HIV 1 and anti HIV 2+p24), syphilis (TP Ab), HTLV-1 and HTLV-2 (Anti HTLV 1 and 2). Tissues are always processed and transported under sterile conditions and under Good Manufacturing Practice (GMP) conditions. For transport, tissues are packed in a primary sterile packaging, a shipping container that is inserted into a secondary sterile packaging.

Step a), i.e. the step of cleaning and resection of the perinatal tissue, is performed after separating the desired tissues from the other tissues of the placenta, especially by separating the amnion from the chorion by sectioning using sterile scissors or a scalpel. Cleaning is carried out by washing (lavage) in a physiological solution and subsequent mechanical cleaning, i.e. manual removal of blood residues and impurities from the tissue spread out on the work surface. The lavage and mechanical cleaning are repeated at least twice, preferably three times. The last lavage is preferably carried out in sterile water for injections. The cleaning and processing of perinatal tissues can be done manually or automatically or semi-automatically.

After the cleaning step, the tissue is allowed to air-dry for at least 15 minutes (still in an aseptic environment), then fragmented and/or shaped into sections no larger than 50 mm in any dimension, preferably no larger than 30 mm in any dimension. The material is inserted into a primary packaging and frozen at a temperature of −40 to −80° C. The material is preferably stored in one dose in a tissue volume of 1 to 100 ml.

The thus processed tissue allows to exploit maximum yield and effect of the following cryo-grinding step.

Within the framework of creating this invention, the inventors tested alternative options for performing step a). A 30-minute lavage with the antibiotic gentamicin resulted in an unacceptable content of the antibiotic in the final product. Larger dimensions of the tissue sections led to uneven cryo-grinding. Excessive water content of the tissue led to problems with cryo-grinding as well as with introducing the tissue into the primary packaging-water was getting into the packaging material, which can lead to a risk of sterility violation.

The packaged tissue is stored for the prescribed quarantine period, which is at least 2 weeks.

Step b), i.e. the cryo-grinding step, is carried out in closed capsules, preferably made of surgical steel, cooled from the outside with liquid nitrogen. A tissue for grinding (without the addition of water) and sterilized cryo-grinding balls made of steel are placed in the capsule, the capsule is closed and placed in a liquid nitrogen cooling jacket, and cryo-grinding is performed. Cryo-grinding is preferably carried out in a cryogenic mill with pre-cooling with shaking at a frequency of at least 3 Hz and with grinding at a frequency of at least 20 Hz, in at least 4 grinding cycles, for a total duration of the grinding cycles of at least 15 minutes. The sample is shaken at a frequency of at least 3 Hz during inter-cooling cycles, so that the tissue does not stick and freeze to the capsule wall.

Within the framework of the development of this process, grinding with the addition of water was also tested, but it resulted in insufficient grinding and in tissue degradation. Sterile water for injections is thus added only after grinding the sample, when it facilitates the removal of the ground sample from the capsule with almost no loss. After the tissue is ground, the container is allowed to thaw and the contents is then mixed with sterile water for injection at a volume ratio of tissue to water ranging from 1:0.7 to 1:1.

Capsules coated on the inside with a layer of zirconia have also been tested, but zirconia is subject to wear and may contaminate the ground tissue. Therefore, grinding capsules and balls made of surgical steel, which are less susceptible to wear, were chosen.

Compared to conventional procedures, pre-cooling during shaking was added to prevent the tissue from freezing to the capsule wall, and the total grinding time was increased. The sample did not come into contact with liquid nitrogen, as this is also undesirable, and especially during contact with liquid nitrogen, the components of the sample would degrade and possibly become contaminated with bacteria that may be present in the nitrogen.

As part of the testing and development of steps b), c) and d), it was made possible to mix (pool) tissues from multiple donors, while in step c) the uniformity and homogeneity of the cryo-grinding product is achieved. Preferably, a product from 3 to 15 donor perinatal tissues is combined.

The homogenization step (step c)) ensures an even mixing of tissue from several donors. Step c) is necessary to achieve product uniformity. It is performed with an ultrasonic homogenizer, preferably at a power of 40 to 100 W, with 10 to 20 pulses. This method of homogenization prevents heating of the ground tissue.

Prior art procedures carry out cryo-grinding only after lyophilization, but within the scope of the present invention it was found that it is more appropriate and efficient to carry out cryo-grinding and homogenization first, and only then to lyophilize the thus obtained material.

Step d) of filling into the primary packaging involves calculating the filling volume by taking multiple samples to measure the dry matter content and then, based on the average dry matter content, calculating the volume of the ground tissue material that corresponds to the required amount of dry matter for filling into the primary package. Preferably, the amount of dry matter for filling into the primary packaging is in the range of 35 to 50 mg/vial of the primary packaging. In some embodiments, this step involves filling the homogenized tissue in a precisely determined volume of 1.3 ml±10% per vial of primary packaging and subsequently checking the filled volume. The determined volume was chosen on the basis of long-term process development and analysis of measured data.

The material is preferably filled into the primary packaging using a peristaltic pump while mixing the material with a magnetic stirrer. The mixture should be constantly aloft, but without the formation of vortex.

The primary packaging is preferably vials.

The subsequent lyophilization step (step e)) is carried out at the lowest pressure value of 100 m Torr (13 Pa) and the lowest temperature of −60° C., preferably at the lowest temperature of −50° C., and then the primary package is closed. Preferably, the temperature drop is gradual, e.g. in the order of hours.

The thus lyophilized material has a very low water content, which extends shelf life, reduces the risk of microorganism growth should contamination occur at any step prior to application of the material, and results in a loose powder that is easier to handle.

The sterilization step (step f)) is performed using gamma radiation. However, the commonly used dose values of 25 to 30 kGy degraded the product so much that it could not even be resuspended. However, it was found that lower doses of radiation ensure the same quality of sterilization, and preserve the function of the proteins, but do not cause the mechanical degradation observed at usual doses. The sterilization step therefore takes place at doses of 5 to 20 kGy, more preferably 12 to 18 kGy.

Furthermore, the result of the process according to the present invention is a powdered material based on perinatal tissue, which can be obtained by the described process. The material preferably has a particle size of no more than 200 μm, but preferably at least 95% of the particles are up to 5 μm in size.

Mass spectroscopy confirmed that this resulting material contained more than 68 proteins. These proteins include cytokines, embryonic growth factors, enzymes, extracellular matrix proteins, hormones, matrix metalloproteinases, neurotrophic factors, protease inhibitors.

More specifically, these proteins include:
Cytokines: TIMP-4, bFGF, PDGF-AA, HGF, EGF, VEGF; glyceraldehyde-3-phosphate dehydrogenase, annexin 2; interleukins (IL): IL-4, IL-6, IL-8, IL-10, IL-13; galectin-1, galectin-3, macrophage migration inhibition factor, pentraxin-3, galectin-7, transforming growth factor beta-induced protein;
Embryonic growth factors: dickkopf-related protein 3 agrin;
Enzymes: 5′-nucleosidase/CD73, alpha-enolase, fructose-bisphosphate aldolase A, glucosidase 2 beta subunit, glutathione S-transferase P, lactotransferrin, neutral alpha-glucosidase AB, peptidyl-prolyl cis-trans isomerase B, peroxiredoxin-1, peroxiredoxin-6, phosphoglycerate kinase 1, phosphoglycerate mutase 1, alpha-1 subunit of prolyl 4-hydroxylase, protein disulfide isomerase, protein disulfide isomerase A3, protein disulfide isomerase A6, transglutaminase 2 (Protein-glutamine gamma-glutamyltransferase 2), transketolase, triose phosphate isomerase 1, ubiquitin-conjugating enzyme E2 N, catalase peroxiredoxin 1 (thioredoxin peroxidase 1), peroxiredoxin 2 (thioredoxin peroxidase 2), superoxide dismutase [Cu—Zn], superoxide dismutase [Mn], mitochondrial precursor, transthyretin precursor (Prealbumin), lactoylglutathione lyase;
Extracellular matrix proteins: collagen I, collagen III, collagen IV, collagen V, collagen VI, collagen VII, fibronectin, laminin alpha-5 subunit, laminin beta-1 subunit, laminin beta-2 subunit, laminin gamma-1 subunit, lumican, nidogen-1, nidogen-2, alpha-3 subunit of laminin, gamma-2 subunit of laminin, beta-2 subunit of laminin, perlecan (basement membrane-specific heparan sulfate proteoglycan core protein);
Hormones: angiotensinogen;
Matrix metalloproteinases: collagenase type IV (matrix metalloproteinase-2), stromelysin-2 (matrix metalloproteinase-10), extracellular matrix metalloproteinase inducer/basigin; Neurotrophic factors: neuron-specific enolase;
Protease inhibitors: alpha-1-antitrypsin, alpha-2-macroglobulin, cystatin B, inter-alpha-trypsin inhibitor heavy chain H1, inter-alpha-trypsin inhibitor heavy chain H2.

The material shows no signs of cytotoxicity towards mammalian cells, has a high binding capacity towards mammalian cells forming a colloidal environment; reduces macrophage-mediated activation compared to activated control cells, without immunomodulatory effects, and has proven anti-inflammatory effects.

Furthermore, the object of the present invention is a pharmaceutical formulation containing material based on perinatal tissue, obtainable by the method according to the invention, and at least one auxiliary substance selected from the group of solvent, gelling agent, propellant, ointment base, cream base, filler, and binder. The pharmaceutical formulation can be, for example, in the form of an injectable solution, suspension, colloid, spray, hydrogel, ointment, cream. Pharmaceutical formulations can be prepared, for example, by dissolving or suspending the powdered material in a solvent, or by combining the powdered material with a propellant to create a spray, or by introducing the powdered material into a gelling agent and reacting to create a hydrogel, or by introducing the powdered material into an already formed hydrogel, or by introducing the powdered material into an ointment or cream base.

The object of the present invention is also the powdered material based on perinatal tissue or the pharmaceutical formulation. Both the powder material and the pharmaceutical formulation are suitable for use in the treatment of inflammatory and degenerative diseases of the musculoskeletal and connective tissues and skin, burns, autoimmune skin disorders, and especially for the treatment of osteoarthritis by intra-articular administration, treatment of tendinitis by local administration, treatment of skin defects (thermal, chemical, mechanical, of trophic or bacterial origin) by topical application, including application in the form of a spray, hydrogel or suspension.

The procedure for the production of a biological therapeutic product according to the invention allows to fully utilize the biological and physical properties of perinatal tissues (e.g. human amniotic membrane, placenta, chorion, umbilical cord), to safely preserve the maximum proportion of natural bioactive components present in the human amniotic membrane, thereby achieving the best distribution in the damaged organ and a larger effective surface during therapeutic administration, and this also ensures the improvement of the function of musculoskeletal and connective tissues, organs and skin. The powder preparation obtained by the process of the invention can be easily reformulated into other pharmaceutical forms, for example, injectable, gel, etc. This allows a greater choice and a possible combination of treatment procedures.

### BRIEF DESCRIPTION OF DRAWINGS

FIG. 1: Comparison of particle size distribution in different regions of the homogenized sample (example 1)

FIG. 2: Comparison of the effect of radiation doses and irradiation temperature on the amount of total protein (example 1)

FIG. 3: Comparison of the effect of radiation doses and irradiation temperature on the amount of interleukins (example 1)

FIG. 4: Activation of macrophages with the product of Example 1.

FIG. 5: Detection of nitrite in the medium.

FIG. 6: Activation of macrophage IL-6 production by the product of Example 1.

### EXAMPLES OF CARRYING OUT THE INVENTION

### Example 1: Preparation of Amnion-Based Material

### Obtaining the Starting Material, Cleaning, Resection:

Placentas are obtained only from healthy donors during a planned cesarean birth. After aseptic collection and informed consent of the donor, placenta is transported under sterile conditions to the GMP laboratory. The placenta is wrapped in a sterile packaging. During processing, the amnion is typically separated from the chorion by sectioning using sterile scissors or a scalpel. The amnion cleaning step consists of two steps. The amnion is gently washed in the physiological solution which is then followed by mechanical cleaning, which is carried out by spreading the amnion on the work surface and manually removing the residual blood and dirt with a gentle movement of the thumb and forefinger. These steps are repeated at least twice. The last cleaning step is lavage in sterile water for injection. After cleaning, the tissue is allowed to air dry (under aseptic conditions), usually for 15 to 40 minutes. The amnion cleaned in this way is stored in a shape not exceeding 50 mm in any dimension, in primary, secondary and tertiary packaging and is frozen in a deep-freeze box.

### Cryo-Grinding:

In the premises of purity class A, the tissue is unpacked and placed in a grinding capsule, and sterilized grinding balls are added. Capsules and grinding balls are made of stainless steel. The capsule is tightly closed and placed in a cooling jacket, which is also tightly closed. Subsequently, a cryo-grinding process is carried out, during which the amnion is micronized. First, pre-cooling is performed with shaking at a frequency of 5 to 10 Hz, then 5 grinding cycles at 20 to 40 Hz, while the grinding cycles are interspersed with inter-cooling cycles at 5 to 10 Hz. This is followed by thawing of the capsule. Cooling is ensured by the flow of liquid nitrogen.

Homogenization and Filling into Primary Packaging:

After grinding the amnion, the container is allowed to thaw and the contents is then mixed with sterile water for injection at a tissue: water ratio of 1:0.8 by volume. The mixture is transferred to a sterile bottle using a sterile disposable pipette and the entire volume is homogenized using an ultrasonic homogenizer, which at 40 to 100 W with 10 to 20 pulses enables uniform homogenization of the entire mixture. The product processed in this way is referred to as intermediate product 1 (MP1).

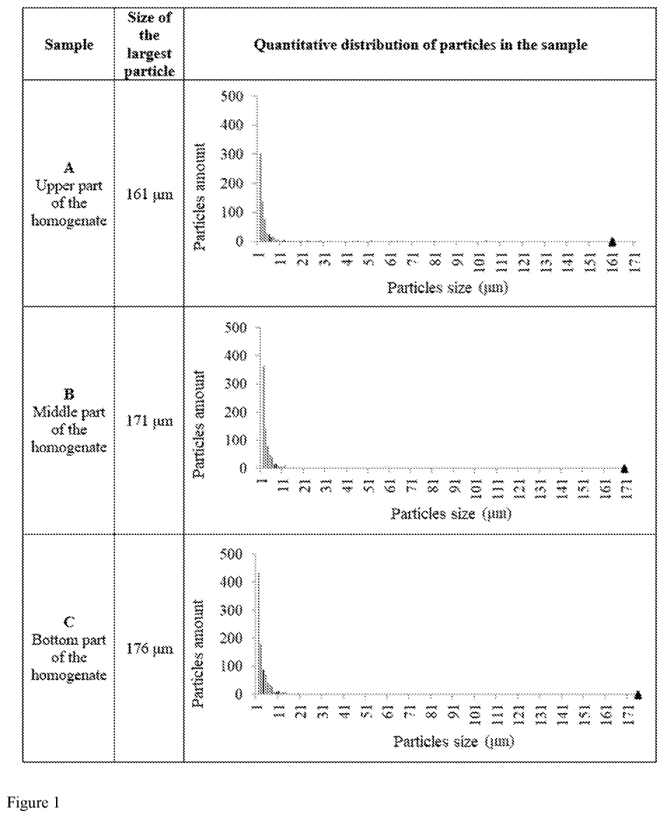

After homogenization, the particle size distribution (PSD) was compared. A comparison of the PSD in different regions of the homogenized sample is shown in FIG. 1.

Furthermore, it was verified that the homogenization does not reduce the content of total protein in the mixture. After cryo-grinding, a sample of the non-homogenized mixture was taken for total protein analysis. Total protein content was measured using the Bradford total protein content method. The samples used for the method are derived by isolating proteins using RIPA buffer. This test is used to measure the concentration of total protein in a sample. The principle of this test is that the binding of protein molecules to the Coomassie Brilliat Blue dye under acidic conditions leads to a change in color from brown to blue. The sample is applied to a microtiter plate in a volume of 5 μl and 250 μl of Coomassie dye is added to it. Subsequently, the plate is read at a wavelength of 595 nm. The values are calculated based on the calibration curve (Standards A-I). Table 1 shows the results of protein determination according to Bradford, including the calibration curve.

TABLE 1
Measured values of protein in the samples by the Bradford method

| | | Average | |
|---|---|---|---|
| | Concentration | absorbance | Standard |
| Sample | μg/ml | value | deviation |
| standard A | 2000 | 0.2733 | 0.0014 |
| standard B | 1500 | 0.1967 | 0.0061 |
| standard C | 1000 | 0.1290 | 0.0042 |
| standard D | 750 | 0.0793 | 0.0060 |
| standard E | 500 | 0.0530 | 0.0067 |
| standard F | 250 | 0.0173 | 0.0026 |
| standard G | 125 | 0.0070 | 0.0067 |
| standard H | 25 | 0.0017 | 0.0031 |
| standard I | 0 | 0.0000 | 0.0000 |
| non-homogenized sample 1 | 1736 | 0.2412 | 0.0012 |
| non-homogenized sample 2 | 1724 | 0.2395 | 0.0027 |
| homogenized sample 1 | 1715 | 0.2389 | 0.0021 |
| homogenized sample 2 | 1685 | 0.2348 | 0.0025 |

Filling from a mixture of amnions from different donors into the primary packaging in a volume of 1.3 ml±10% is carried out with a peristaltic pump or another way so that the mixture is constantly in a float. After filling, the vials are equipped with a rubber stopper, which is in such a position that air can flow into the vial during the subsequent step.

### Lyophilization:

The next step is freeze drying (lyophilization), which dries the already micronized mixture of amnion and water. After lyophilization, the product is cooled to a temperature of 5±3° C. An example of a lyophilization protocol is shown in Table 2.

TABLE 2
Parameters of the lyophilization protocol

| | Programmed parameters | | |
|---|---|---|---|
| | Shelf temperature | | |
| Step in lyophilization | (° C.) | Time (min) | Vacuum (Pa) |
| Temperature | 5 | 30 | N/A |
| adjustment | 0 | 15 | N/A |
| | −10 | 90 | N/A |
| | −15 | 30 | N/A |
| | −45 | 320 | N/A |
| Freezing temperature | −45 | N/A | N/A |
| Freezing time | N/A | 10 | N/A |
| Vaccum start | −60 | N/A | 53 |
| Primary drying | −20 | 1260 | 13 |
| Secondary drying | 10 | 30 | 60 |
| Storage | 5 | N/A | 60 |

### Sterilization:

Vials with the lyophilized material are provided with sterile alloy caps under sterile conditions. After this step, the product is transported to final sterilization. The final sterilization is carried out in a facility certified for the sterilization of biological therapeutic products at doses of 5 to 20 kGy (using the VDmax15 method), when the obtained product was a loose powder.

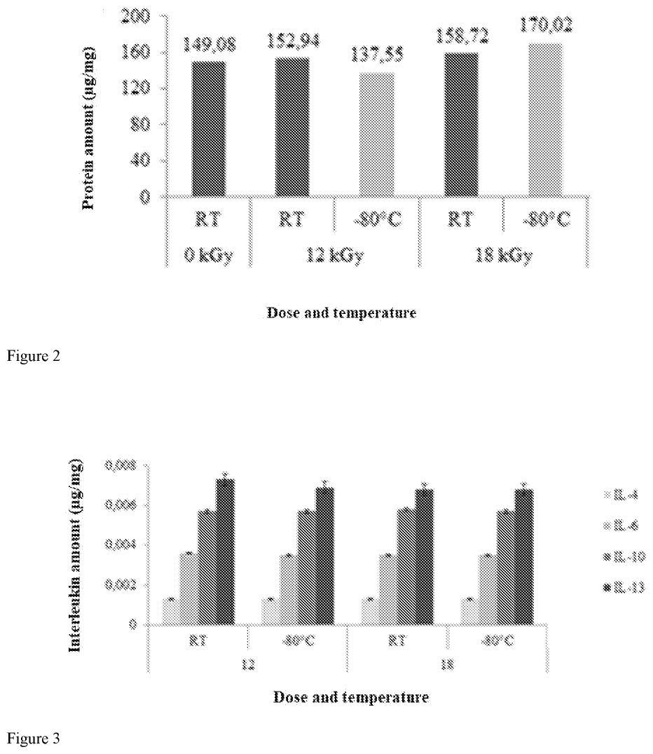

The total protein content of the thus obtained product was determined by the Bradford method. The samples used for the method are derived by isolating proteins using RIPA buffer. Furthermore, the content of interleukins was determined with a commercial kit using the ELISA method. The results are shown in FIGS. 2 and 3 and show that sterilization at the indicated doses did not reduce protein or interleukin content. The sterilization temperature had no effect on the content of these substances either. When the sterilization was carried out at doses of 25 to 30 kGy, the result was a solid phase that was sticky, gooey, and no further experiments could be performed with it.

### Product Characterization:

Tests and studies have been carried out on the product obtained by the described procedure showing the stability for transport, storage and biological activity.

Product stability was monitored for 4 weeks. The water content was determined by coulometric Karl Fischer titration with thermal desorption, the total protein content by the Bradford method. The results are shown in Table 3.

TABLE 3
Product stability results after 4 weeks

| Week | Residual water content [%] | Total protein [ug/mg] |
|---|---|---|
| 0 | 1.743 | 154.633 |
| 2 | 1.8 | 173.152 |
| 4 | 1.8 | 158.397 |

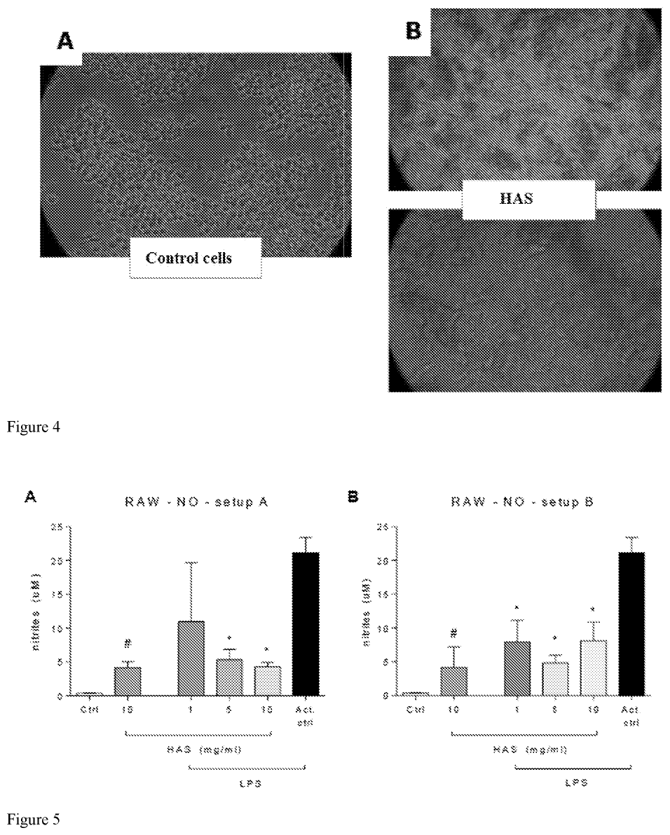

Cytotoxicity testing was performed using several in vitro mammalian cell cultures. Untreated (control) macrophages are shown in FIG. 4A. Culture of macrophages treated with the product (10 mg/ml) demonstrated adherence of the test material to cells and colloid formation in FIG. 4B. Immunomodulatory activity of human placental samples was tested using standard cell lines of mouse macrophages RAW 264.7. THP-1—human and undifferentiated cells of the monocytic lineage and a complex system of whole human blood were used. Immunomodulatory activity was studied by detecting various activation markers, i.e. endotoxin from E. coli (LPS—lipopolysaccharide)—activation of RAW cells and long-term activation of human blood; ii. TNF-α-activation of THP-1 cells. The product shows no signs of cytotoxicity. It also has a high binding capacity towards mammalian cells forming a colloidal environment. The results are shown in FIG. 4.

Nitrite detection was carried out by the Griess reaction. NO production is one of the highly sensitive macrophage activation markers. Data for activated samples were analyzed by statistical ANOVA and compared with activated control followed by Dunnett's multiple comparison test. The product contains traces of nitrites, which is typical for amniotic membranes. Product application (before and after macrophage activation) significantly reduced cell mediated activation compared to the activated control without product application. This demonstrates the immunomodulatory and anti-inflammatory potential of the product. The results are shown in FIG. 5 (HAS=product).

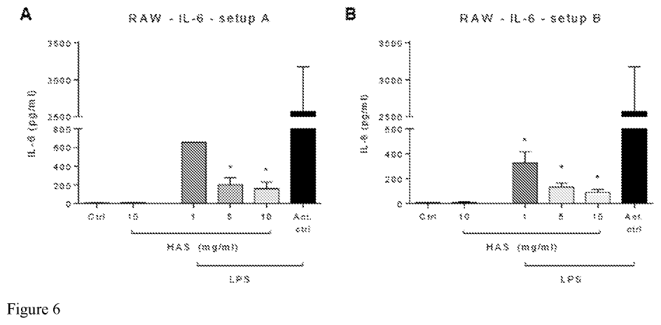

Furthermore, the production of interleukin 6 was investigated. IL-6 is a typical pro-inflammatory cytokine produced, for example, by activated macrophages. Setting A-pretreatment with the product and further activation, setting B-cell activation and subsequent treatment with the product. In both cases, the pretreatment was 1 h and the entire treatment was 24 h. Data were expressed as mean+standard error of the mean. A one-sample t-test was used to analyze the significance of the product treatment with the untreated control. Data for activated samples were analyzed by ANOVA, compared with activated control and followed by Dunnett's multiple comparison test. Macrophage activation and IL-6 secretion by RAW cell lines with/without cultivation in the presence of the product was tested using an ELISA assay. The product significantly reduced macrophage IL-6 production after LPS activation in various experimental settings. The results are shown in FIG. 6 (HAS=product).

### Example 2: Preparation of Material Based on Perinatal Tissue

### Obtaining the Starting Material, Cleaning, Resection:

Placentas are obtained only from healthy donors during a planned cesarean birth. After aseptic collection and informed consent of the donor, the placenta t is transported under sterile conditions to the GMP laboratory. The placenta is wrapped in a sterile packaging. During processing, all types of perinatal tissues are separated from each other using sterile scissors or a scalpel. The perinatal tissue cleaning step consists of two steps-gentle washing in physiological solution and mechanical cleaning. These steps are repeated at least twice. The last cleaning step is lavage in sterile water for injection. After cleaning, the tissue is allowed to air dry (under aseptic conditions), usually for 15 to 40 minutes. The tissue cleaned in this manner is stored, in a shape not exceeding 50 mm in any dimension, in primary, secondary and tertiary packaging and is frozen in a deep-freeze box.

### Cryo-Grinding:

In the premises of purity class A, the tissue is unpacked and placed into a grinding capsule, and sterilized grinding balls are added. Capsules and grinding balls are made of stainless steel. The capsule is tightly closed and placed in a cooling jacket, which is also tightly closed. Subsequently, the cryo-grinding process is carried out. First, pre-cooling is performed with shaking at a frequency of 5 to 10 Hz, then 5 grinding cycles at 20 to 40 Hz, while the grinding cycles are interspersed with inter-cooling cycles at 5 to 10 Hz. This is followed by thawing of the capsule. Cooling is ensured by the flow of liquid nitrogen.

Homogenization and Filling into Primary Packaging:

After grinding the tissue, the capsule is allowed to thaw and the contents is then mixed with sterile water for injection at a tissue: water ratio of 1:0.85 by volume. The mixture is transferred to a sterile bottle using a sterile disposable pipette, the entire volume is homogenized using an ultrasonic homogenizer, which at 40 to 100 W and 10 to 20 pulses enables uniform homogenization of the entire mixture, and the mixture is filled into the primary packaging in a volume of 1.3 ml±10%.

Similar to Example 1, the volume of tissue for dispensing into vials is calculated, and dispensing is also performed as in Example 1.

### Lyophilization:

The next step is freeze drying (lyophilization), which dries the already micronized mixture of perinatal tissues with water. The same lyophilization protocol as in Example 1 can be used.

### Sterilization:

Sterilization is carried out similarly to example 1, with a dose of 5 to 20 kGy of gamma radiation (by the VDmax15 method). A loose powder was obtained.

The tested reference standard dose of 25 to 30 kGy resulted in a product that was sticky and unanalyzable. Such a product would be problematic for use due to uncertain solubility and protein degradation.

Mass spectroscopy of the preparation sterilized with a dose of 12 to 18 kGy gamma radiation confirmed that this resulting material contained more than 68 proteins. Proteins contained and identified include:
Cytokines: glyceraldehyde-3-phosphate dehydrogenase, annexin 2, TIMP-4, bFGF, PDGF-AA, HGF, EGF, VEGF, cytokines IL-4, IL-6, IL-8, IL-10, IL-13, galectin-1, galectin-3, macrophage migration inhibition factor, pentraxin-3, galectin-7, transforming growth factor beta-induced protein; Embryonic growth factors: dickkopf-related protein 3 agrin;
Enzymes: 5′-nucleosidase/CD73, alpha-enolase, fructose-bisphosphate aldolase A, glucosidase 2 beta subunit, glutathione S-transferase P, lactotransferrin, neutral alpha-glucosidase AB, peptidyl-prolyl cis-trans isomerase B, peroxiredoxin-1, peroxiredoxin-6, phosphoglycerate kinase 1, phosphoglycerate mutase 1, alpha-1 subunit of prolyl 4-hydroxylase, protein disulfide isomerase, protein disulfide isomerase A3, protein disulfide isomerase A6, transglutaminase 2 (Protein-glutamine gamma-glutamyltransferase 2), transketolase, triose phosphate isomerase 1, ubiquitin-conjugating enzyme E2 N, catalase peroxiredoxin 1 (thioredoxin peroxidase 1), peroxiredoxin 2 (thioredoxin peroxidase 2), superoxide dismutase [Cu—Zn], superoxide dismutase [Mn], mitochondrial precursor, transthyretin precursor (Prealbumin), lactoylglutathione lyase;
Extracellular matrix proteins: collagen I, collagen III, collagen IV, collagen V, collagen VI, collagen VII, fibronectin, laminin alpha-5 subunit, laminin beta-1 subunit, laminin beta-2 subunit, laminin gamma-1 subunit, lumican, nidogen-1, nidogen-2, alpha-3 subunit of laminin, gamma-2 subunit of laminin, beta-2 subunit of laminin, perlecan (basement membrane-specific heparan sulfate proteoglycan core protein);
Hormones: angiotensinogen;
Matrix metalloproteinases: collagenase type IV (matrix metalloproteinase-2), stromelysin-2 (matrix metalloproteinase-10), extracellular matrix metalloproteinase inducer/basigin;
Neurotrophic factors: neuron-specific enolase;
Protease inhibitors: alpha-1-antitrypsin, alpha-2-macroglobulin, cystatin B, inter-alpha-trypsin inhibitor heavy chain H1, inter-alpha-trypsin inhibitor heavy chain H2.

The material has a particle size of no more than 200 μm.

The product can be further formulated as needed into the form of injectable preparations (dissolved in water for injection), gel preparations (introduced into a gel base), ointments (introduced into an ointment base), creams (introduced into a cream base). Ointment, cream and gel bases are commonly commercially available, for example from the supplier Ambiderman.

### Characterization of the Effectiveness and Safety of the Product:

Collagen, which forms the main component of the extracellular matrix and is an abundant part of the product, was chosen as the primary indicator of the effectiveness of the product.

The primary effectiveness of the collagen in the product can be further supported by the natural occurrence of growth factors that induce the regeneration of joint cells and were therefore chosen as additional indicators of the effectiveness of the product. These are Epidermal Growth Factor (EGF) and Platelet-Derived Growth Factor (PDGF), which induce tissue regeneration. The third selected indicator of effectiveness is Tissue Inhibitor of Metalloproteinases 1, TIMP1, which plays an important chondroprotective role.

The minimum amount of collagen was determined according to the measurement to be 500 to 680 μg/mg, which is 50 to 68% of the product's weight.

As for growth factors, the measured values of the EGF content in the preparation are around 2700 μg/ml. Since a decrease in EGF level by approximately 150 μg/ml was detected in OA patients, we conclude that a single intra-articular dose of the product will be able to increase the EGF level beyond the physiological value.

The concentration of PDGF-BB in the product, based on the carried out tests, ranges from 78.4 to 131 μg/ml. These values correlate with the values reported in the literature, where the PDGF value was quantified in the range of 53 to 119 μg/ml.

The range of TIMP1 values in the product is according to the performed analyses, 204 to 323 ng/ml. Such a concentration is sufficient to increase the overall level of TIMP-1 in the joint and effectively inhibit metalloproteinases, thus mitigate the further progression of OA.

Interleukin 6 (IL-6) was originally chosen as the active substance of the product. However, due to the fact that its increased values are correlating with a poor prognosis of OA, it is likely that the increased value of IL-6 in the preparation may result in a decrease in its effectiveness. For this reason, IL-6 was finally determined as a criterion for product safety.

Based on studies of literature and on the basis of the measured values of individual indicators in the product, the acceptance criteria listed in Table 4 were established for all analyzed components in the product.

TABLE 4
Analyzed components of the preparation

| Analyzed component | | Acceptance criteria | |
|---|---|---|---|
| Primary | Total kolagen | 500-800 | μg/mg |
| effectiveness | | | |
| criterion | | | |
| Auxiliary | Total protein | 110-300 | μg/mg |
| effectiveness | EGF | 200-350 | pg/mg |
| criteria | PDGF-BB | 6-12 | pg/mg |
| | TIMP-1 | 15000-30000 | pg/mg |
| Safety criterion | IL-6 | ≤5 | pg/mg |

1. A method for preparation of a biological therapeutic product based on perinatal tissue, comprising the steps of:
a) cleaning of the starting perinatal tissue and/or shaping into sections whose dimensions do not exceed 50 mm in any direction; and allowing the tissue to dry for at least 15 minutes after the cleaning;
b) cryo-grinding of the perinatal tissue in resealable surgical steel capsules with surgical steel grinding balls;
c) homogenization of the ground perinatal tissue using an ultrasonic homogenizer;
d) transferring the ground and homogenized perinatal tissue into a primary packaging, with constant stirring;
e) lyophilization of the ground and homogenized perinatal tissue in the primary packaging; and
f) sterilization of the lyophilized ground and homogenized perinatal tissue in the primary packaging by gamma radiation at a dose of 5 to 20 kGy, wherein the entire process is carried out without the presence of antibiotics and antimycotics.

2. The method according to claim 1, wherein the step of cleaning and resection of the perinatal tissue is carried out by lavage in a physiological solution and subsequent mechanical cleaning, including manual removal of blood residues and impurities from the tissue, whereby the lavage and mechanical cleaning are repeated at least twice.

3. The method according to claim 1, wherein the cryo-grinding step is carried out in a cryogenic mill by pre-cooling with shaking at a frequency of at least 3 Hz and by grinding at a frequency of at least 20 Hz, in at least 4 grinding cycles, for a total duration of grinding cycles of at least 15 minutes, wherein during inter-cooling cycles the perinatal tissue is shaken at a frequency of at least 3 Hz.

4. The method according to claim 1, wherein the homogenization step is performed with an ultrasonic homogenizer at a power of 40 to 100 W, with 10 to 20 pulses.

5. The method according to claim 1, wherein the ground and homogenized perinatal tissue is filled into the primary packaging using a peristaltic pump while stirring the ground and homogenized perinatal tissue.

6. The method according to claim 1, wherein the lyophilization step is performed at the lowest pressure value of 13 Pa and the lowest temperature of −60° C., and the primary packaging is subsequently closed.

7. A pharmaceutical formulation comprising a powdered material based on perinatal tissue, obtained by the method according to claim 1.

8. The pharmaceutical formulation comprising a powdered material according to claim 7, which has a particle size of no more than 200 pm, with at least 95% of the particles having size of up to 5 μm, and which contains cytokines, embryonic growth factors, enzymes, extracellular matrix proteins, hormones, matrix metalloproteinases, neurotrophic factors, protease inhibitors.

9. A pharmaceutical formulation comprising a powdered material based on perinatal tissue obtained by the method according to claim 1, and at least one auxiliary substance selected from the group of solvent, gelling agent, propellant, ointment base, cream base, fillers and binders.

10. The pharmaceutical formulation according to claim 9 for the treatment of inflammatory and degenerative diseases of musculoskeletal and connective tissues and skin, burns, autoimmune skin disorders, skin defects.

11. The method according to claim 2, wherein the last lavage is carried out in sterile water for injection.

12. The method according to claim 5, wherein the ground and homogenized perinatal tissue is stirred with a magnetic stirrer.

13. The pharmaceutical formulation according to claim 9 for the treatment of osteoarthritis by intra-articular administration, tendinitis by local administration, or for the treatment of skin defects by topical application.
